(19) United States
(12) Patent Application Publication
(10) Pub. No.: US 2017/0135934 A1
(43) Pub. Date: May 18, 2017

(54) Mouth Rinse for Prevention of Periodontal Disease

(71) Applicant: Henry Mallek, Washington, DC (US)

(72) Inventor: Henry Mallek, Washington, DC (US)

(21) Appl. No.: 14/944,334

(22) Filed: Nov. 18, 2015

(51) Int. Cl. A61K 8/64; A61K 8/37; A61Q 11/00; A61K 8/49; A61K 8/34; A61K 8/86; A61K 8/60

(52) U.S. Cl. CPC A61K 8/64; A61K 8/86; A61K 8/37; A61K 8/60; A61K 8/49; A61K 8/34; A61K 8/602; A61Q 11/00; A61K 2800/74

(57) ABSTRACT

The present invention causes the tissues in the gingiva to be less permeable to the products of bacterial metabolism produced from dental plaque. In addition, chemicals derived from the ingestion of foods and beverages are blocked from entry into the gingival tissues. This results in the prevention of oral infections and, most importantly, the loss of teeth. In addition to the ingress of toxic chemicals, this mouth rinse results in cessation of gingival crevicular fluid, a medium for bacteria to thrive on. The overall result is a healthy periodontium which is free of infected tissues and inflammation and bodes well for optimum oral and systemic health.

### BACKGROUND OF THE INVENTION

Nutrition plays critical roles in the physiology of the periodontium. This can be understood by considering each periodontal tissue and its state of metabolism individually. The arrangement of the gingival tissues to the tooth crown and root surfaces is unique, unlike any other clinical situation. The gingival epithelia, which covers the underlying connective tissues, can be categorized into three different types based on their location, composition and structure. The oral epithelium extends from the mucogingival junction to the tip of the gingival crest and is subdivided into the free marginal gingiva and the attached gingiva. The sulcular epithelium lines the gingival sulcus and extends from the tip of the gingival crest to the coronal most portion of the junctional epithelium. The junctional epithelium extends from the base of the gingival sulcus to an arbitrary point approximately 2.9 mm coronal to the alveolar bone crest and is closely adapted to the tooth surface to form sealing and attachment functions. These three epithelia differ ultrastructurally with distinct phenotypic differences in their expressions of cell surface markers.

### Junctional Epithelium

It is commonly accepted that the junctional epithelium exhibits structural and functional features that contribute to preventing pathologic bacterial flora from colonizing the subgingival tooth surface. The junctional epithelium is firmly attached to the tooth surface and thereby forms an epithelial barrier against plaque bacteria. It however, allows the flow of gingival crevicular fluid (GCF) into the sulcus. This is composed of inflammatory cells and components of the immunological host defense which gain access to the gingival sulcular tissues. The junctional epithelium is part of the attachment between the tooth and gingiva and plays an extremely important role in periodontal health and disease. Because it is adapted for adherence to the tooth surface, this epithelium differs from the sulcular and oral epithelia in several respects. In healthy states it is thinner than these other epithelia. The junctional epithelium is 15 to 30 cell layers in its coronal portion and thins to a few cell layers at its termination towards the cementoenamel junction.

The junctional epithelium has several characteristics which provide it with enhanced permeability. The suprabasal cells which are flattened with their long axes running parallel to the tooth surface have relatively few intercellular junctions but have distensible intercellular spaces and the adhesion between the epithelial cells thereby is reduced as compared with other gingival epithelia. These spaces allow the diffusion of tissue fluids from the connective tissue through the epithelium into the gingival sulcus. Also important in terms of periodontal disease etiology, is the lack of keratinization at the epithelial surface, the orientation of cells with their long axes parallel to the tooth surface and the intercellular spaces all of which are factors permitting the passage of bacterial products from the sulcus into the gingival connective tissues.

An important criterion concerning the roles of nutrition in the etiology of periodontal disease and the health of periodontal tissues is the turnover times of these tissues. The junctional epithelial cells exhibit rapid turnover which contributes to the host-parasite equilibrium and rapid repair of damaged tissues. The junctional epithelium is constantly renewed by mitotic division of basal cells, coronal migration, and sloughing from the surface at the base of the gingival sulcus. The turnover time of these cells being only 4 to 6 days. The number of desquamated cells per surface area unit is many times higher than elsewhere in the oral mucosa. The significance of this is the prominent role of nutrition in the physiology of the periodontium. Cells which are turning over rapidly are more affected by nutritional deficiency than those which are not. Putting it another way-a, nutrient deficiency is less likely to affect cells with a long turnover time.

Previously it was thought that only epithelial cells facing the external basal lamina were rapidly dividing. However, recent evidence indicates that a significant number of DAT (dental attached cells) cells are, like the basal cells along the connective tissue, capable of synthesizing DNA, which demonstrates their mitotic activity. At the coronal part of the junctional epithelium, the DAT cells typically express a high density of transferrin receptors which supports the idea of their active metabolism and high turnover. The findings suggest that the DAT cells have a more important role in tissue dynamics and reparative capacity of the junctional epithelium than has been previously thought. The .existence of a rapidly dividing population of epithelial cells (DAT cells) in a suprabasal location, several layers from the connective tissue is a unique feature of the junctional epithelium. This distinct phenotype may result from specific permissive or instructive signals provided by the internal basal lamina matrix on the tooth surface. Therefore any structural or molecular changes in the internal basal lamina can potentially influence the vital functions of the DAT cells and contribute to the effectiveness or failure of the junctional epithelial defense. Changes in cell metabolism which can be caused by nutrition, significantly affect the internal basal lamina and the cells of the junctional epithelium.

The junctional epithelial cells are different than other epithelial cells. This is due to the fact that unlike other epithelial cells they are attached to both soft (connective tissues) and hard tissues, namely the tooth surface. This requires a dual function and an epithelial cell that has to adapt to two different environments and may make these cells vulner able to the action of different exogenous and endogenous factors or agents. Nutrition requirements are critical in such situations.

Although the internal basal lamina morphologically appears to be relatively resistant to external challenges its molecular structures may still be altered, leading to changes in DAT cell function. For example, it has been shown that certain matrix metalloproteinases from eukaryotic cells cleave lamin-5, exposing a cryptic molecular site that triggers cell migration. It has not been shown however, if bacterial proteinases can directly perform the same selective cleavage of laminin-5. Bacterial agents may thus indirectly trigger mechanisms that lead to modulation of host cell behavior. Hypothetically, even minor changes in cell metabolism, biosynthetic activity or ability to divide and migrate may eventually lead to degeneration and detachment of the junctional epithelium/DAT cells and allow pathogenic flora to grow on the exposed subgingival tooth surface. A wide variety of bacterial species and their products have been shown to adversely affect the metabolism of epithelial cells leading to changes in proliferation and production of cytokines and matrix metalloproteinases. Deficiencies or excesses of nutrients may modulate the junctional epithelium to withstand these effects of bacterial products.

The junctional epithelium consists of active populations of cells and, with antimicrobial functions, together form the first line of defense against microbial invasion. Even though junctional epithelial cell layers provide a barrier against bacteria many bacterial substances such as lipopolysaccharides pass through the epithelium and pass through the external basal lamina into the connective tissue. Both the internal and external basal laminas can however, act as barriers against infective agents.

Rapid turnover is an important factor in the microbial defense of junctional epithelium. Also, because the area is covered by the dividing cells in the junctional epithelium it is at least 50 times larger than the area through which the epithelial cells desquamate into the gingival sulcus; providing a strong funneling effect that contributes to the flow of epithelial cells. Rapid shedding and effective removal of bacteria adhering to epithelial cells is an important aspect of the antimicrobial defense mechanisms at the dentogingival junction and is significantly affected by nutrition.

Inflammatory mediators and antibodies produced by macrophages, lymphocytes and plasma cells in the gingival tissues restrict the spreading of bacterial infection into the connective tissue and systemic circulation. Significant numbers of lymphocytes may be present also within the junctional epithelium, thus contributing to the protective function of this tissue. Also, supplementary to system-derived antibodies and antibodies produced locally by plasma cells, the junctional epithelial cells has a secretory function.

### Glutamine Roles in Periodontal Metabolism

Glutamine is the most abundant free amino acid in the body. Intracellular glutamine concentration varies between 2 and 20 mM (depending upon cell type) whereas its extracellular concentration averages 0.7 mM. Glutamine plays an essential role, promoting and maintaining function of various organs and cells such as kidney, intestine, and other epithelium, liver, heart, neurons, lymphocytes, macrophages, neutrophils, pancreatic B-cells, and adipocytes. At the most basic level, glutamine serves as an important fuel in these cells and tissues. A high rate of glutamine uptake is characteristic of rapidly dividing cells such as enterocytes, fibroblasts and lymphocytes where glutamine is an important precursor of peptides and proteins as well as of amino sugars, purines, and pyrimidines, thus participating in the synthesis of nucleotides and nucleic acids. Glutamine metabolism additionally provides precursors for the synthesis of key molecules such as glutathione (GSH). Glutamine supplementation attenuates glutathione depletion. Glutamine affects gene expression where it has been shown that approximately 1% of 10,000 genes assessed by microarray techniques were altered by addition of glutamine. Changes in gene expression, impact on cell function which means a change in glutamine concentration, will alter many clinical parameters. In cultures of neutrophils recovered from burnt and postoperative patients, addition of glutamine augmented the in vitro bacterial killing activity and was also important for the production of reactive oxygen species (ROS). There is no doubt that neutrophils play an important role in oral defense of the periodontium and that glutamine affects the functions of these cells.

The importance of glutamine for cell function is firmly established. Glutamine is a source of respiratory fuel and nitrogen for biosynthetic reactions in addition to playing diverse regulatory roles in cell physiology. It is quantitatively the most important fuel for a number of rapidly dividing cells and tissues such as the junctional and sulcular epithelium. It is metabolized to L-alanine in epithelial cells by a route involving conversion to glutamate, then 2-oxoketoglutarate via glutaminase and glutamate dehydrogenase, then TCA cycle conversion to malate followed by the action of NADP-dependent malic enzyme to create pyruvate which undergoes amination to produce L-alanine via the action of alanine aminotransferase.

Glutamine plays an important role in gluconeogenesis in liver, kidney and many different tissues. It can function as a substrate and also controls the expression and activity of phosphoenolpyruvate carboxykinase (PEPCK) a key regulatory enzyme of gluconeogenesis. Several studies in humans have shown that in the post absorptive state, glutamine is an important glucose precursor and makes a significant contribution to the addition of new carbon to the glucose pool. Glutamine is an important substrate of the urea cycle, is utilized as a precursor for lipid synthesis in adipocytes and counteracts glucocorticoid-induced muscle atrophy by preventing the decline in myosin heavy chain synthesis.

Glutamine plays an important role in cell proliferation of cells such as lymphocytes and enterocytes. Proliferation of Caco-2 cells was increased by nucleoside, nucleotide and glutamine supplementation but not by glutamate. Arginine potentiated the effect of glutamine. The effects of glutamine and arginine on Caco-2 cell proliferation are mediated by the stimulation of nucleotide synthesis and that the major role of glutamine in this process was not energy supply. The synthesis of both purines and pyrimidines was stimulated by the administration of extracellular glutamine.

Glutamine activates both ERKs and JNKs(extracellular signal regulated kinase and Jun kinase, respectively),proteins involved in signal transduction pathways stimulated by growth factors in IEC-6(epithelial cells from rat small intestine) and IPEC-J2 (porcine intestine epithelial cell line) cells, resulting in an increase in AP-1 dependent gene transcription and c-Jun mRNA levels. AP-1 and c-Jun are transcription factors that regulate the expression of genes involved in cell division. Glutamine potentiates the effects of growth factors on cell proliferation and repair.

Glutamine serves as an energy source for tissues such as the heart. It increases the synthesis of collagen in human fibroblasts by a direct stimulatory effect and as a precursor of proline and hydroxyproline residues. The direct effect of glutamine on collagen biosynthesis includes a dose-dependent increase in transcription and mRNA steady state of collagen. The response of fibroblasts with respect to collagen synthesis and mRNA reached a maximal level at glutamine concentrations between 0.15 and 0.25 mM and did not change further up to 10 mM. Glutamine is involved in the synthesis of ECM proteins in cultured mesangial cells. Glutamine at 2 mM elicited an increase in type IV collagen and fibronectin transcripts compared to control cells in the absence of glutamine. Such effects on collagen synthesis also occur in the gingiva which is dominated by collagen.

Glutamine plays an important role in cell defense and repair which is evident in its role as a potent enhancer of heat shock protein 72(HSP72). Heat shock at 43 degrees C. induces intestinal epithelial cell death, as reflected by a marked increase in floating cell count. In the absence of glutamine supplementation, the percentage of floating cells reached 90%. Supplementation of glutamine at concentrations higher than 1 mM caused a dose dependent decrease in the percentage of floating cells.

Glutamine is a potent enhancer of heat shock protein 72(HSP72) expression in vitro and in vivo. The induction of a heat shock can attenuate pro-inflammatory cytokine release. HSP may down regulate cytokine expression binding to the heat shock element present in the promoter region of interleukin 1B(1L1b) and potentially of other cytokines, a process that results in downregulation of cytokine expression.

Intravenous glutamine dipeptide administration in septic rats has resulted in significantly lower mortality compared to those on conventional diets. Human studies have reported that glutamine treated patients experienced fewer clinical infections and shorter hospital stays. Therefore glutamine is essential in the treatment of established infections or inflammation as occurs in the periodontium.

A recent development in the metabolic support of critically ill patients has been the evolution of the concept called “immunonutrition.” Nutrients have been identified that stimulate cells in such a manner as to enhance immunologic responses and potentially improve outcome. The amino acid glutamine is included in the list of “immunonutrients” that possess these biological effects. While it is true that glutamine has immunoenhancing properties, the overall benefit derived from this amino acid may arise because it is an essential nutrient and immune modulation is only one facet of its essential nature.

Glutamine is a required nutrient for cell growth in tissue culture. Eagle reported that both mouse fibroblasts and HELa cells degenerated and died unless the cell medium was supplemented with glutamine. When glutamine was added in increasing concentration there was a dose response increase in cell proliferation. Glutamine could not be replaced by glutamic acid or glucose.

The proliferative response of rat and human lymphocytes by uptake of radioactive thymidine is dependent on the presence of glutamine. Proliferation of rat lymphocytes as quantified by radioactive thymidine was four-fold when glutamine was added to the medium, effects not seen with other amino acids or ammonia. Many studies have demonstrated that glutamine is an essential fuel and substrate for support and differentiation of human lymphocytes. In addition to supporting cell growth, glutamine serves as an important substrate which enhances other functions of immunological cells. For example the rate of phagocytosis in macrophages has a close relationship with glutamine levels in the medium.

Glutamine affects neutrophil function; when studied in vitro; addition of glutamine improves bactericidal function. With glutamine, neutrophils are better able to kill Staphylococcus aureus. Glutamine restores neutrophil function to supranormal levels. Glutamine is essential for immune system cells to differentiate and function in normal ways. Glutamine acts as an energy source for proliferating cells; it provides essential nutrients for nucleic acid synthesis, and supplies the nitrogen necessary for the formation of glycosamines and other cell intermediates. Also important, this amino acid may serve as a regulatory signal which, by itself or in conjunction with other growth factors, indicate that cell proliferation can proceed.

### Glutamine Dipeptides-Tissue Specific Nutrients

Much research has been conducted on the assumption that “tailor-made nutrition solutions” will increase the benefits of intravenous nutrition for specific patient groups. Amino acid mixtures for the treatment of renal and liver disease are examples of such developments. Considerable interest is now being devoted to the development of new substrates containing nutrients such as amino acids. Recent knowledge concerning the efficient utilization of intravenously supplied dipeptides and tripeptides creates the possibility of substituting short chain peptides for available amino acid solutions.

A profound and seminal research development was reported and conducted by Matthews (Matthews, 1972) in London and Adibi (Adibi, 1987) in Pittsburgh who reported that amino acids could be absorbed more rapidly from peptides than in free form. Few theories in physiologic research have evoked as much interest as that of intact peptide absorption Accordingly, in the last 20 years, peptide absorption and subsequent utilization have been the subjects of intensive investigation. The discovery of an active transport system for uptake of dipeptides and tripeptides was the first important stage in the understanding of the process of this new progressive research field. This peptide carrier system facilitates subsequent intracellular hydrolysis of dipeptides and tripeptides by peptide hydrolases. Peptidases are widely distributed in intracellular and extracellular compartments of mammalian tissues and the bulk of activity resides in the cytoplasm.

Current research in biochemistry and medicine has resulted in reconsideration of classification of essential and nonessential amino acids. In healthy adults, cysteine and glutamine are considered dispensable amino acids because they can be synthesized readily from precursor substrates. Profound intramuscular glutamine depletion is a characteristic feature of catabolic conditions and malnutrition. The extent of glutamine depletion is unrelated to the magnitude of stress or nutritional intervention. Glutamine supports rapidly proliferating immune cells, serves as an energy source for the gastrointestinal and other mucosa and immunologically active tissues and provides carbon and nitrogen precursors for the biosynthesis of nucleotides and phospholipids. Amino acids such as cysteine, tyrosine and especially glutamine are considered as conditionally indispensable amino acids and should be constituents of nutrition support. The instability of glutamine in aqueous solutions prevents its addition to parenteral solutions or mouth washes in adequate amounts. There is a growing body of evidence that the use of stable and highly soluble glutamine in the form of synthetic dipeptides resolves the problem of how to formulate and prepare adequate solutions to be used in clinical nutrition.

It has been convincingly demonstrated that supplementation with glutamine improves nitrogen balance, enhances the rate of protein synthesis, supports immune cells, and maintains integrity of the mucosa, especially epithelium. It is also been proven that provision of glutamine prevents translocation of bacteria and toxins from the intestinal tract and other mucosa to the general circulation.

Two unfavorable chemical properties of free glutamine hamper its use in routine clinical setting; instability especially during heat sterilization and prolonged storage and limited solubility (−3 g/100 ml at 20 degrees C.). The rate of breakdown of free glutamine depends on temperature pH, and anion concentration. This decomposition of free glutamine is quantitative and yields the products pyroglutamic acid and ammonia. Several studies have shown that free glutamine may be provided by adding the crystalline amino acid to a commercially available amino acid solution before administration. However appropriate preparation of such a solution requires a daily procedure at +4 degrees C. under strict aseptic conditions in the local pharmacy and subsequent laborious sterilization by membrane filtration. In addition to diminishing the risk of precipitation, the glutamine concentrations in such solutions should not exceed 1.0-1.5%. Consequently, provision of adequate amounts of free glutamine to injured or critically ill patients represents a severe burden, especially in volume restricted situations. Thus the parenteral or oral use of free glutamine is reserved only for controlled and well conducted clinical trials.

The implication of stable and highly soluble synthetic dipeptides shows great promise as a route for the provision of amino acids otherwise difficult to deliver. Dipeptides with glutamine, tyrosine, cysteine, and taurine residues at the C-terminal position reveal high solubility in water and sufficient stability during heat sterilization and prolonged storage. These properties qualify the dipeptides to be approved as suitable constituents of liquid nutritional preparations. Since the 1950s there is convincing evidence for the intact absorption of dipeptides. There is a mode of absorption of protein digestion products which involves mucosal uptake of peptides followed by intracellular hydrolysis. In 1968, Matthews in London and Adibi in the U.S. reported that amino acids are absorbed more rapidly from peptides than in the free form.

Dipeptide analogues of glutamine such as L-Alanyl-L-gutamine (AlaGln) have been developed to overcome the problems associated with poor solubility and instability. Glutamine dipeptides with C-terminal glutamine are highly soluble in water and sufficiently stable during heat sterilization and prolonged storage.

Basic studies with various synthetic glutamine-containing short chain peptides provide convincing evidence that these new substrates are rapidly cleared from plasma after parenteral administration without being accumulated in tissues and with inconsequential losses in the urine. Significant hydrolase activity in extra- and intracellular tissue compartments ensures a quantitative peptide hydrolysis, the liberated amino acids being available for protein synthesis, generation of energy, and significant, beneficial effects on the epithelium and the immune system.

Following bolus injection or under conditions of continuous parenteral nutrition (TPN) or in an oral rinse, these peptides provide glutamine for the maintenance of the intra- and/or extracellular pool. Addition of L-Alanyl L-glutamine as a stable glutamine source to a standard TPN solution or mouth rinse enhances mucosal cellularity and function and reverses atrophy-associated gut and mucosal dysfunction in parenterally or oral fed rats and humans. In isolated segments of distal ileum of piglets, intravenous endotoxin infusion (50 g/kg) was associated with increased permeability. The endotoxin-induced permeability change could be prevented or significantly delayed by supply of luminal glutamine. Supplemental glutamine was associated with improved survival and lower degree of bacterial translocation in experimental sepsis of gut origin. Monosaccharide transport, water absorption, and mucosal morphology are preserved with AlaGln enriched TPN following an experimental two-step small bowel transplantation procedure. It is concluded that glutamine is essential for physiological absorptive and barrier function of intestinal grafts. Direct intraluminal infusion of glutamine into the graft improves mucosal structure and absorption of D-xylose.

The postulate that glutamine or glutamine dipeptides exert a beneficial effect on the mucosa is strongly supported by the results of a current study in which biopsies from normal human ileum, proximal colon and rectosigmoid colon were incubated with glutamine, AlaGln and saline. Glutamine and AlaGln equally stimulated crypt cell proliferation; the trophic effect was mainly confined to the basal crypt compartments.

Glutamine supply and endogenous arginine production are related. Compared to a control diet, glutamine enrichment resulted in higher arterial plasma concentration of citrulline and arginine in experimental animals. It is conceivable that supplemental glutamine caused increased renal arginine production from citrulline. This effect of glutamine supplementation is beneficial considering the multiple, important biological properties of arginine.

Human studies in healthy volunteers demonstrated that AlaGln is readily hydrolyzed after its bolus injection; the elimination t1/2 ranging between 3 and 4 minutes. Continuous infusion of a commercial amino acid solution supplemented with AlaGln or glycyl-L glutamine (GlyGln) was not accompanied by any side effects and no complaints were reported. Infusion of the peptide-supplemented solutions resulted in a prompt increase in alanine, glutamine and glycine concentrations. During the entire infusion period, only trace amounts of the dipeptides could be measured in plasma, and urinary losses of dipeptides were only about 1% of the given dose. These results indicate a “nearly “quantitative hydrolysis” of the infused peptide and subsequent utilization of the constituent free amino acids. Lochs et al. (Lochs, 1990) studied the organ clearance of glutamine-containing dipeptides in postabsorptive and starved humans. The clearance of AlaGln and GlyGln by the kidney was greater than those measured for the splanchnic or skeletal muscle. Infusion of the dipeptides was associated with increased plasma concentrations and enhanced splanchnic uptake of glutamine and alanine or glycine, respectively.

The first clinical study with a synthetic dipeptide was performed in 1987 in patients undergoing elective resection of the colon or rectum. Infusion of AlaGln-supplemented TPN over 5 days resulted in an improvement of nitrogen balance on each postoperative day compared with controls receiving isonitrogenous and isoenergetic TPN without the peptide The improved net nitrogen balance was associated with maintenance of the intracellular glutamine pool, whereas in patients receiving the control solution, glutamine levels were markedly decreased compared with preoperative values. The peptide was not detectable in plasma and the plasma concentrations of the constituent amino acids did not differ between the treatment groups. The infusion of the solutions was free of any side effects and postoperative recovery was normal for each patient. In good agreement with these results, intravenous supply of AlaGln following cholecystectomy preserved the intracellular glutamine pool (91% of postoperative value) and the characteristic postoperative decline in muscle ribosomes was abolished.

Petersson et al. (Patersson et al., 1991) studied the long term effect of postoperative TPN supplemented with GlyGln on protein synthesis in skeletal muscle. In the glutamine group, the decrease in protein synthesis (assessed by ribosome profiles) was less pronounced compared with controls. Beneficial effects of short-term infusion of AlaGln on muscle protein synthesis assessed by 14Cleucine incorporation were reported by Barua et al. (Barua et al., 1992) in postsurgical patients receiving glutamine-free parenteral nutrition. In patients undergoing major abdominal operations the beneficial effects of glutamine dipeptide-supplemented TPN on nitrogen economy, lymphocyte recovery, maintenance of plasma glutamine concentration and shortened hospital stay was observed. A novel finding was the striking influence of supplemental glutamine dipeptide on cysteine-leukotriene (Cys-LT) metabolism. After operation, the low Cys-Lt concentration in isolated polymorphonuclear leukocytes was completely restored with supplemental dipeptide while remaining low with conventional TPN. Cys-LT's are potent lipid mediators. It has been emphasized that diminished release of these mediators is accompanied with an attenuated endogenous host defense, which is of great concern in periodontal tissues which are highly prone to infection.

There are numerous studies emphasizing the immunostimulatory role of supplemental glutamine dipeptides. Increased counts of circulating total lymphocytes and enhanced T-cell lymphocyte synthesis are consistently found in stressed patients following provision of glutamine or glutamine dipeptide-containing nutrition.

As in animal experiments, it could be demonstrated that glutamine dipeptide-containing TPN may avoid trauma-related intestinal atrophy, associated with glutamine-free TPN. In patients with inflammatory bowel disease and neoplastic disease, intestinal permeability could be maintained and villus height preserved with GlyGln supplementation. In another study, AlaGln-supplemented TPN maintained absorptive capacity (assessed by D-xylose absorption test) in the proximal portion of the small intestine in critically ill patients, compared with patients receiving conventional glutamine-free TPN. Because the large intestine harbors far more bacteria than the duodenum, jejunum, or ileum, the maintenance of an intact colonic barrier is crucial. In this context the trophism of glutamine and the stable dipeptide AlaGln on the colonic mucosa is clinically relevant.

It is conceivable that during stress and especially in critical illness, and infection, antioxidant capacity is decreased due to formation of free radicals. Glutamine supplementation has been shown to preserve hepatic glutathione and intestinal mucosal glutathione stores. In a current study, the combined therapy with vitamin E and glutamine was successful in the treatment of a severe veno-occlusive (VOD disease) following BMT. It can be assumed that glutamine (dipeptide) supplementation can contribute to replenishment of depleted glutathione stores during stress and thereby counteract free radical-induced cellular injury. Indeed these protective mechanisms combined with benefits to the immune system, plays a major role in influencing morbidity and outcome.

In conclusion glutamine is considered to be a conditionally indispensable amino acid during stress. Available data indicate that glutamine is an important amino acid in a number of clinical settings. Indeed, omission of glutamine from conventional TPN and its subsequent supplementation should be considered as a replacement of a deficiency rather than a supplementation. It is conceivable that the beneficial effects observed with glutamine nutrition is simply a correction of disadvantages produced by an inadequacy of conventional amino acids.

### SUMMARY OF THE INVENTION

Although bacteria in the form of dental plaque are considered to be the prime etiologic factor in periodontal disease, nutrition is the principal modulator of the resistance of the host to the harmful effects of oral microorganisms. The investigator of this patent is highly trained and qualified in the fields related to this subject. He is a dentist and periodontist who studied the specialty of periodontics at Tufts University where he studied under the foremost investigators in the field. In addition, he holds a PH.D. in Nutritional Biochemistry and Metabolism from the Massachusetts Institute of Technology (MIT). His qualifications in the area of nutrition and periodontal tissue metabolism are without equal.

The purpose of this invention is eradication of periodontal disease, and improvement of oral and systemic health. This is accomplished by using a mouth rinse twice a day. Unlike any other mouth rinse, it contains the amino acid glutamine in the form of a dipeptide which makes the periodontal tissues impermeable to the harmful toxic chemicals in dental plaque. The oral health derived from the use of this mouth rinse obviates the need of periodontal surgery to treat this disease. Most importantly, the mouth rinse is safe, pleasant to use and eliminates oral malodors.

### DETAILED DESCRIPTION OF THE INVENTION

It has been discovered that there is a new method to prevent the harmful effects of oral bacteria in the mouth and avoid the destruction that these cause to the gums, teeth all oral tissues and organs of the body. This invention shuts down the permeability of the gingiva around the teeth. In so doing, bacteria and toxic substances cannot pass into the gingiva. This keeps the gingiva in a state of health as well as the whole body.

When the gingiva is healthy, that is, it is free of infection, this results in healthy bone supporting the teeth. This is very important, because the principle problem with gum disease is the loss of bone which occurs from the spread of infection from the gingiva to the underlying bone around the teeth. In fact, the major cause of tooth loss in adults is loss of bone caused by infection in the gingiva. When severe bone loss occurs, the teeth become mobile and fall out of the jaws.

A major factor of gingival inflammation is gingival fluid that oozes from the gingival pocket. This fluid is produced by the inflamed soft tissue wall of the pocket and ranges from a clear serous liquid to a highly viscous pus or purulent exudate. Gingival fluid is primarily composed of inflammatory cells mostly polymorphonuclear leucocytes and serum proteins. In addition, the fluid contains bacteria, tissue breakdown products, enzymes, antibodies, complement, and a wide variety of inflammatory mediators. The amount and rate of fluid production at a given site are highly variable and correlates with the severity of inflammation.

When gingival fluid was discovered, it was thought to be a fluid that merely passed from the blood into the gingival sulcus. It was assumed that it was a continuous flow of liquid substance that cleansed the sulcus. Additional studies however, showed that the output of fluid had to be provoked and that the fluid resembled an exudate rather than a transudate of plasma. Also important, was the observation that the amount of collectable fluid increased with the extent and severity of gingival inflammation.

Although it has been substantiated that many different types of cells migrate through the junctional epithelium, what is most important in terms of the etiology of periodontal disease and gingival defense is that foreign materials and fluids pass through the gingival epithelium and enter the underlying connective tissue. Relatively large molecules like albumin, antigens and a number of enzymes may penetrate the junctional and sulcular epithelium. Many other enzymes such as histamine, hyaluronidase and horseradish peroxidase may increase the size of the intercellular spaces and make the epithelium more permeable to bacterial toxins and other toxic substances.

Since bacteria found in bacterial plaque produce such enzymes, gingival defense may be lowered by bacterial enzymes making the junctional and sulcular epithelium permeable to toxic substances that would not otherwise penetrate these epithelia.

The position of the gingiva is crucial to understanding the disease process affecting the teeth and how this invention prevents disease of the gingiva and teeth and prevents their loss. Around each tooth there is a shallow grove between the tooth and the gingiva. Microscopic measurements on cross sections of the marginal region have shown that the depth of the sulcus is normally 0.5 mm or less and becomes much deeper as the tissues become more inflamed. The bottom of the sulcus is formed by the free surface of the junctional epithelium whereas the coronal portion is lined by an epithelium which is the sulcular termination of the oral sulcular epithelium. The demarcation between junctional and oral sulcular epithelium is very distinct. In a normal and ideal condition only few polymorphonuclear leucocytes can be observed within the intercellular spaces of the junctional epithelium. In other words unlike the oral sulcular epithelium, the junctional epithelium which does not have a keratin cover is permeable to chemicals present around the teeth and most importantly in the sulcus of the gingiva.

Bacteria play a major role in the etiology of inflammatory periodontal disease. Enzymes, endotoxins, cytotoxic metabolic products and immune reactions are among the active chemicals related to bacteria. On the other hand direct invasion of the periodontal tissues by bacteria has not been found except in one disease, acute necrotizing ulcerative gingivitis (ANUG).

Transport through the gingival epithelium which is comprised of the oral sulcular and junctional epithelium takes place in both directions. An example is an enzyme produced by oral bacteria, horseradish peroxidase. Not only does this chemical reach the underlying connective tissue but it also widens the spaces between the gingival epithelial cells. Albumin is an example of another molecule which is large in size but can easily penetrate the sulcular epithelium.

In addition to agents synthesized by oral bacteria which affect the permeability of the gingiva, the resident cells of this area produce permeability enhancing chemicals. An example of these is the enzyme collagenase. Various studies have shown this enzyme produced in inflamed gingival tissues breaks up or lyse collagen the main structural protein of the periodontal tissues. Inasmuch collagen is broken down or destroyed during the course of periodontal disease, this effect of the enzyme collagenase is a major detrimental agent in the pathogenesis of periodontal disease.

In its role as a barrier between the external environment and the periodontium, the junction between the tooth and the gingiva is subject to a wide range of environmental factors. In addition to the chemicals produced by oral bacteria found in plaque, these include extremes in temperature and pH accompanying food intake, saliva, and gingival crevicular fluid.

In addition to changes brought about in the epithelium of the sulcus and the junctional epithelium, chemicals produced by either the tissues, bacteria or food produce harmful changes in the internal environment of the gingiva resulting in a change in the rate of cellular division consistent with changes observed in early or initial periodontal disease.

Glutamine an amino acid plays a pivotal role in preventing the harmful effects of chemicals in the tissues of the gingiva whether they be from the epithelium, connective tissues, bone or from the bacteria in the form of dental plaque. This is accomplished by rinsing the mouth with the mouth rinse of this invention. This mouth rinse has the following composition:

Sorbitol

Poloxomer 407

Sodium saccharin

Propylparaben

Sucralose

Methylparaben

Alanyl-L-glutamine

Glycyrrhizic acid

Menthol

Deionized water

As a result of the effects of this mouth rinse, the gingiva is protected from the effects of harmful chemicals produced by bacteria. This is attributable to the mouth rinse shutting down the permeability of the junctional and sulcular epithelium. Also, the mouth rinse protects the gingiva from the effects of similar chemicals such as collagenase produced by the oral tissues.

Because these harmful chemicals produced by bacteria or oral tissues cause bone loss around the teeth, the blocking of their harmful effects prevents loss of teeth in addition to dental decay.

The oozing of fluid from the gingiva as a result of oral infection has deleterious effects on gingival and systemic health. Use of this mouth rinse significantly improves the health of the periodontium because it shuts down and decreases the flow of gingival fluid and its toxic substances.

By blocking the effects of harmful chemicals on the periodontal tissue the severity of periodontal infections is significantly reduced. A number of studies have pointed to systemic effects of periodontal disease. Use of this mouth rinse has important general health benefits such as affecting the progression of such diseases as arthritis cardiovascular disease and diabetes.

Due to the beneficial effects on periodontal health, gingival bleeding and tissue hyperplasia is eliminated. These effects, in addition to lower levels of plaque, eliminate oral malodor.

### References

Roth, E., Glutamine:An Anabolic Effector. JPEN 13: 1990:130S-140S. Lochs, H., et. al Splanchnic, Renal, and Muscle Clearance of Alanylglutamine in Man. Metabolism 39: 1990: 833-836.

Van Der Hulst, R. W. J. Glutamine and the Preservation of Gut Integrity. Lancet 341: 1993: 1363-1365.

Tamada, H., et al., The Dipeptide Alanyl-glutamine Prevents Intestinal Mucosal Atrophy in Parenterally Fed Rats. JPEN 16: 1992: 110-116.

Hollander, D., The Intestinal Permeability Barrier. Scand J Gastroenterol. 1992: 27:721-726.

Lo, C W., Walker, W A., Changes in the Gastrointestinal Tract During Enteral or Parenteral Feeding. Nutr Rev. 1989: 47: 195-198.

Goeters, C., et. al, Parenteral 1-Alanyl-L-Glutamine Improves 6-Month Outcome in Critically Ill Patients. Crit Care Med 2002: 30: 2032-2036.

Abumrad, N N., et al. Possible Sources of Glutamine for Parenteral Nutrition: Impact on Glutamine Metabolism. Am J Physiol 243: E123-E131, 1982.

Furst, P., Albers, S., Stehle, P. Glutamine-Containing Dipeptides in Clinical Nutrition. JPEN 14: 118S-124S, 1990

Meister, A., Metabolism of Glutamine. Physiol Rev. 36: 103-127. 1956.

Carneiro-Filho. B A., et. al. Alanyl-Glutamine Hastens Morphologic Recovery from 5-Fluorouracil-Induced Mucositis in Mice. Nutrition 2004; 20: 934-941.

Ikeda, S et al. Glutamine Improves Impaired Cellular and Polymorphonuclear Neutrophil Phagocytosis Induced by Total Parenteral Nutrition after Glycogen-Induced Murine Peritonitis. Schock 2003: 19: 50.

Salvalaggio, P R., Campos, A C., Bacterial Translocation and Glutamine. Nutrition. 2002: 18:435.

Higashiguchi, T., et al. Effect of Glutamine on Protein Synthesis in Isolated Intestinal Epithelial Cells. JPEN 1993: 17: 307-314.

Souba, W W. et al. Oral Glutamine Reduces Bacterial Translocation Following Abdominal Radiation. J Surg Res. 1990: 48:1-5.

Furst, P. et al. Reappraisal of Indispensable Amino Acids. Ann Nutr Metab. 1997: 41: 1-9.

Babst, R., et al. Glutamine Peptide-Supplemented Long Term Total Parenteral Nutrition: Effects on Intracellular and Extracellular Amino Acid Patterns, Nitrogen Economy and Tissue Morphology in Growing Rats. JPEN. 1993; 17:566-574.

Windmuellar, H G. Glutamine Utilization by the Small Intestine. Adv Enzymol 1982:53:202.

Lowe, D K., et al. Safety of Glutamine-enriched Parenteral Nutrient Solutions in Humans. Am J Clin Nutr. 1990:52: 1101-1106.

Stehle, P., Kuhne, B., Kubin, W., Furst, P. Synthesis and. Characterization of Tyrosine and Glutamine-containing Peptides. J Appl Biochem 1982:4:280.

Lochs, H., Williams, P E., Morse, E L., Abumrad, N N., Adibi, S A. Metabolism of Dipeptides and their constituent Amino Acids by Liver, Gut, Kidney and Muscle. Am J Physio. 254: (Endocrinol Metab.17) E588-E594, 1988.

Stallard, R E., Awwa, I A. The Effect of Alterations in External Environment on the Dentogingival Junction. J. Dent Res Supplement, 1969: 48: 671-675.

Mandel, I D., Dental Plaque: Nature Formation and Effects. J Periodont 1966: 37: 357-367:

Schultz-Haudt, S, Effect of Hyaluronidase on Human Gingival Epithelium. Science 1965; 117: 653-655.

Brandtzaeg, P. The Significance of Oral Hygiene in the Prevention of Dental Disease. Odont T. 1964: 72: 460-486.

Thilander, H. The Efect of Leukocyte Enzyme Activity on the Structure of the Gingival Pocket Epithelium in Man. Acta Odont Scand 1963: 21: 431-450.

Caffesse, R G., Nasjleti, C E. Enzymatic Penetration Through Intact Sulcular Epithelium J Periodontol. 1976: 47: 391-397.

Socransky, S S. Relationship of Bacteria to the Etiology of Periodontal Disease. J. Dent Res 1970: 49: 203.

Tolo, K J. A Study of Permeability of Gingival Pocket Epithelium to Albumin in Guinea Pigs and Norwegian Pigs. Archs Oral Biol 1971: 16:881-888.

McDougall, W A. Penetration Pathways of a Topically Applied Foreign Protein into Rat Gingiva. Periodont Res. 1971:6: 89-99.

Furst, P., Stehle, P. The Potential Use of Parenteral Dipeptides in Clinical Nutrition. Nutr Clin Pract 1993:8: 106-114.

Platell. C., McCaully R., McCulloch, R., Hall, J. Influence of Glutamine and Branched Chain Amino Acids on the Jejunal Atophy Associated with Parenteral Nutrition. J Gastroenterol Hepatol 1991:6: 345-349.

Vazquez, J A. et al. Dipeptides in Parenteral Nutrition: from Basic Science to Clinical Application. Nutr Clin Pract. 1993:48: 95-105.

Tabak, L A et al: Role of Salivary mucins in Protection of the Oral Cavity. J Oral Pathol. 1982:11: 1-17.

Katsuragi, Y. et al. Basic studies for the Practical Use of Bitterness Inhibitors: Selective Inhibition pf Bitterness by Phosopholipids. Pharm Res 1997: 14: 720-724.

Kroeze, J H A., Bartoshuk, L M. Bitterness Suppression as Revealed by Split-Tongue Taste Stimulation in Humans. Physiol Behav. 1985: 35: 779-783.

Matthews, D M. Intestinal Absorption of Peptides. Physio. Rev. 1975: 55: 537-608.

Linden, S K., Sutton, P., Karlsson, N G., Karolik, V., McGluckin, M A. Mucins in the Mucosal Barrrier to Infection. Mucosal Immunology. 2008: 1: 183-197.

Reitzer, L j., Wice, B M., Kennel. D. Evidence that Glutamine, not Sugar, is the major Energy Source for HeLa cells. J Biol Chem. 1979. 254; 2669-2676. 1979.

Furst, P. Pogan, K., Stehle, P. Glutamine Dipeptides in Clinical Nutrition. Nutrition. 1997; 13: 731-737.

Albers, S., Wernerman, J., Stehle, P., Vinnars, E., Furst, P. Availability of Amino Acids Supplied by Constant Intravenous Infusion of synthetic Dipeptides in Healthy Man. Clin Sci. 1989: 76: 643.

Robinson, C., Kirkham, J., Percival, R., et al. A Method for the Quantative site-specific Study of the Biochemistry Within Dental Plaque biofilms Formed in vivo. Caries Res 1997; 31: 194-200.

Herzog, B., Frey, B., Pogan, K., Stehle, P., Furst, P. In Vitro Peptidase Activity of Rat mucosal Fractions against glutamine containing Dipeptides. J nutr. Biochem. 1996: 7:135.

Hammarqvist, F., et al. Alanyl-Glutamine Counteracts the Depletion of Free Glutamine and the Postoperative Decline in Protein synthesis in Skeletal Muscle. Ann Surg. 1990:212: 637-644.

Barua, J M., Wilson, E., Downie, S., Weryk. B., Cushieri., A., Rennie, M J. The Effect of Alanyl-glutamine peptide supplementation on muscle Protein Synthesis in Post Surgical Patients Receiving Glutamine Free Amino Acids Intravenously. Proc Nutr. Soc., 51: 104A, 1992.

Paterssson, B., Waller S-O., Van der Decken, A., Vinnars, E., Wernerman, J., The Long Term Effect of Postoperative TPN Supplemented with glycyl-glutamine on Protein Synthesis in Skeletal Muscle. Clin Nutr. (Suppl 2), 10, 1991.

Adibi, S. A. Experimental Basis for use of Peptides as Substrates for Parenteral Nutrition. Metabolism 36: 1001-1011, 1987.

Matthews, D. M. Intestinal Absorption of Amino Acids and Peptides. Proceedings of the Nutrition Society 31: 171-177, 1972.

What is claimed is:

1. A mouth rinse which blocks the permeability of the gingival tissues to toxic chemicals found in dental plaque and chemicals present in ingested foods and beverages.

2. A mouth rinse comprising reagents including surfactants such as 3% poloxomer 407; preservatives such as 0.5% methylparaben; 0.5% propylparaben; sorbitol; sweeteners such as 1% sucralose and 1% sodium saccharin; flavoring agents such as 0.1-0.2% menthol; 0.1% bitter blocking agent, glycyrrhizic acid; 0.1% active agent alanyl-L-glutamine; and the balance of deionized water.

3. A mouth rinse preparation as set forth in claim 1, said mouth rinse preparation remaining stable upon continuous exposure to a temperature of 37 degree C. at atmospheric pressure for at least 2 years and wherein the pH is at least about 4.5.

4. A mouth rinse preparation in claim 1, said mouth rinse preparation prevents oral and systemic infection and eliminates oral malodor.
